(19) United States
(12) Patent Application Publication
(10) Pub. No.: US 2008/0306564 A1
(43) Pub. Date: Dec. 11, 2008

(54) METHOD AND APPARATUS FOR SHORT-TERM HEART RATE VARIABILITY MONITORING AND DIAGNOSTICS

(75) Inventors: Xuan Wei, Plymouth, MN (US); Gerrard M. Carlson, Champlin, MN (US); Shantha Arcot-Krishnamurthy, Roseville, MN (US)

Correspondence Address: SCHWEGMAN, LUNDBERG & WOESSNER, P.A., P.O. BOX 2938, MINNEAPOLIS, MN, 55402, US

(73) Assignee: Cardiac Pacemakers, Inc, St Paul, MN (US)

(21) Appl. No.: 11/761,107

(22) Filed: Jun. 11, 2007

(51) Int. Cl. A61N 1/365

(52) U.S. Cl. 607/18

(57) ABSTRACT

A diagnostic system monitors autonomic using short term heart rate variability (STHRV). Some examples apply a therapy that is adjusted based on wellness indicator. A wellness indicator is a measure of the STHRV produced to indicate a patient's cardiac condition.

### TECHNOLOGY FIELD

This document generally relates to cardiac rhythm management (CRM) systems and particularly, but not by way of limitation, to a method and apparatus for short-term heart rate variability (STHRV) diagnostics and therapy.

### BACKGROUND

One problem faced by physicians treating cardiovascular patients is assessing patient well-being for providing a prognosis or for adjusting therapy to improve the patient's prognosis. Heart rate variability (“HRV”) is thought to provide one assessment of cardiovascular wellness. The time interval between intrinsic ventricular heart conduction events changes in response to the body's metabolic need for a change in heart rate and the amount of blood pumped through the circulatory system. For example, during a period of exercise or other activity, a person's intrinsic heart rate will generally increase over a time period of several heartbeats. However, even on a beat-to-beat basis, that is, from one heart beat to the next, and without exercise, the time interval between intrinsic heart contractions varies in a normal person. These beat-to-beat variations in intrinsic heart rate are the result of proper regulation by the autonomic nervous system of blood pressure and cardiac output; the absence of such variations indicates a possible deficiency in the regulation being provided by the autonomic nervous system.

### Overview

In Example 1, a cardiac rhythm management system includes a sensing circuit configured to sense a cardiac signal, a short term heart rate variability (STHRV) measurement circuit, coupled to the sensing circuit, configured to characterize the cardiac signal to measure at least a first STHRV measurement during a period of time compatible with an outpatient procedure and to provide at least a first wellness indicator based on the first STHRV measurement and a wellness circuit configured to compute a first wellness indicator using the first STHRV measurement.

In Example 2, system of Example 1 is optionally configured to include a communications circuit coupled to the wellness circuit and a programmer configured to communicate with the communications circuit and to display the wellness indicator.

In Example 3, the system of Example 1 can be optionally configured such that the sensing circuit comprises a circadian rhythm measurement circuit to associate the first wellness indicator with one of a plurality of circadian rhythm parameters.

In Example 4, the system of Example 1 can be optionally configured such that the STHRV measurement circuit comprises one or more of a Standard Deviation of Normal-to-Normal intervals (SDNN) circuit to provide an SDNN characterizing the STHRV measurement, a Standard Deviation of Averages of Normal-to-Normal intervals (SDANN) circuit to provide an SDANN characterizing the STHRV measurement, a Ratio of Low Frequency STHRV to High Frequency STHRV ratio (LF/HF ratio) circuit to provide an LF/HF ratio characterizing the STHRV measurement, an STHRV footprint circuit to provide an STHRV footprint characterizing the STHRV measurement, and a Root-Mean-Square of Successive Differences (RMSSD) circuit to provide an RMSSD characterizing the STHRV measurement.

In Example 5, the system of Example 1 is optionally configured to include a pulse output circuit configured to deliver electrical stimulation pulses and a stimulation control circuit coupled to the sensing circuit, the STHRV measurement circuit, and the pulse output circuit, the stimulation control circuit being configured to control delivery of one or more electrical stimulation pulses based on at least the first wellness indicator.

In Example 6, the system of Example 5 can be optionally configured such that the stimulation control circuit is configured to control delivery of cardiac resynchronization therapy (CRT) pacing.

In Example 7, the system of Example 5 can be optionally configured such that the stimulation control circuit includes a feedback circuit configured to adjust delivery of the one or more electrical stimulation pulses based on the first wellness indicator and at least a second wellness indicator.

In Example 8, the system of Example 1 can be optionally configured such that the period of time is approximately 5 minutes.

In Example 9, the system of Example 8 can be optionally configured such that the period of time is approximately 2 minutes.

In Example 10, a method includes sensing a cardiac signal and measuring STHRV to provide at least a first wellness indicator based on a first STHRV measured during a time period compatible with an outpatient procedure.

In Example 11, the method of Example 10 is optionally configured to include storing the first STHRV and comparing the first STHRV to a threshold STHRV to determine the first wellness indicator.

In Example 12, the method of Example 10 can be optionally configured such that measuring STHRV to provide the wellness indicator comprises measuring STHRV to provide an autonomic balance parameter indicative of a balance between sympathetic and parasympathetic activities.

In Example 13, the method of Example 10 is optionally configured to include detecting sleep apnea by measuring a first respiratory parameter and a second respiratory parameter and associating the first respiratory parameter with the first wellness indicator and a the second respiratory parameter with a second wellness indicator.

In Example 14, the method of Example 10 is optionally configured to include measuring a first posture and a second posture, and associating the first postures with the first wellness indicator, and the second posture with a second wellness indicator.

In Example 15, the method of Example 10 is optionally configured to include measuring a second STHRV to provide a second wellness indicator and trending the first wellness indicator and the second wellness indicator to provide an STHRV wellness trend.

In Example 16, the method of Example 15 is optionally configured to include measuring the first STHRV and the second STHRV under substantially similar conditions and indicating a potential heart failure condition when the STHRV wellness trend demonstrates a decrease in STHRV over time.

In Example 17, the method of Example 16 is optionally configured to include measuring a third STHRV to provide a third wellness indicator and providing an indication of a potential myocardial infarction when the first and third wellness indicators are substantially similar, while the second wellness indicator, that is measured between the first and third wellness indicators, demonstrates a wellness indicator that is less than the first and third wellness indicators.

In Example 18, the method of Example 10 is optionally configured to include detecting a first activity level and associating the first activity level with the first wellness indicator, detecting a second activity level and associating the second activity level with a second wellness indicator and detecting a decrease in wellness when the second wellness indicator is less than the first wellness indicator.

In Example 19, the method of Example 18 is optionally configured to include providing an activity adjustment indicator when a decrease in wellness is detected.

In Example 20, the method of Example 10 is optionally configured to include delivering electrical stimulation pulses based at least on the first wellness indicator.

In Example 21, the method of Example 20 is optionally configured to include associating a first electrical stimulation therapy with the first wellness indicator, measuring a second STHRV to provide a second wellness indicator, associating a second electrical stimulation therapy with the second wellness indicator, trending the first wellness indicator and the second wellness indicator to provide an STHRV wellness trend, determining an improved electrical stimulation based on the STHRV wellness trend and providing the improved electrical stimulation therapy.

In Example 22, the method of Example 20 is optionally configured to include sensing a neural signal, associating the first wellness indicator with the neural signal and delivering neurostimulation therapy based on the association of the first wellness indicator with the neural signal.

In Example 23, a system includes an implantable case including at least a first lead, the first lead having at least a first electrode, means for sensing a cardiac signal, the means for sensing a cardiac signal being disposed in the implantable case and being in electrical connection with at least the first electrode, means for measuring STHRV to provide at least one wellness indicator based on a cardiac signal measured during an outpatient procedure and means for indicating wellness based on associating at least the first wellness indicator with at least a first wellness state.

In Example 24, the system of Example 23 can be optionally configured such that the means for sensing the cardiac signal include a sensing circuit configured to sense a cardiac signal.

In Example 25, the system of Example 24 can be optionally configured such that the means for measuring STHRV to provide at least one wellness indicator based on the cardiac signal during an outpatient procedure include a short term heart rate variability (STHRV) measurement circuit, coupled to the sensing circuit, and configured to characterize the cardiac signal to measure at least a first STHRV measurement during a period of time compatible with an outpatient clinical visit and to provide at least a first wellness indicator based on the first STHRV measurement.

This Overview is an overview of some of the teachings of the present application and not intended to be an exclusive or exhaustive treatment of the present subject matter. Further details about the present subject matter are found in the detailed description and appended claims. Other aspects of the invention will be apparent to persons skilled in the art upon reading and understanding the following detailed description and viewing the drawings that form a part thereof, each of that are not to be taken in a limiting sense. The scope of the present invention is defined by the appended claims and their equivalents.

### BRIEF DESCRIPTION OF THE DRAWINGS

In the drawings, which are not necessarily drawn to scale, like numerals describe similar components throughout the several views. The drawings illustrate generally, by way of example, but not by way of limitation, various examples discussed in the present document.

FIG. 1 is an illustration of an example of a CRM system and portions of an environment in which the CRM system is used.

FIG. 2 is a block diagram illustrating one example of a diagnostic system that is shown with an optional CRM system.

FIG. 3 is a block diagram illustrating one example of a pacing system being part of the CRM system.

FIG. 4 is a block diagram illustrating one example of a neurostimulation system being part of the CRM system.

FIG. 5 illustrates multiple STHRV measurements associated with time.

FIG. 6 illustrates multiple STHRV measurements associated with multiple cardiac resynchronization therapy (CRT) parameters.

FIG. 7A illustrates multiple STHRV measurements associated with multiple activity levels.

FIG. 7B illustrates multiple STHRV measurements associated with respiration and multiple activity levels.

FIG. 8A is a block diagram illustrating one example of a stimulation parameter feedback system.

FIG. 8B is a three dimensional graph illustrating improved CRT pacing based on STHRV measurements.

FIG. 9 is a flow chart illustrating one example of a method for stimulation parameter feedback using a wellness indicator.

### DETAILED DESCRIPTION

The following detailed description includes references to the accompanying drawings, which form a part of the detailed description. The drawings show, by way of illustration, specific embodiments in which the invention may be practiced. These embodiments, that are also referred to herein as “examples,” are described in enough detail to enable those skilled in the art to practice the invention. The embodiments may be combined, other embodiments may be utilized, or structural, logical and electrical changes may be made without departing from the scope of the present invention. The following detailed description is, therefore, not to be taken in a limiting sense, and the scope of the present invention is defined by the appended claims and their equivalents.

In this document, the terms “a” or “an” are used, as is common in patent documents, to include one or more than one. In this document, the term “or” is used to refer to a nonexclusive or, unless otherwise indicated. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

This document discusses, in various examples, a system to measure cardiac wellness. Some examples discussed can include a diagnostic system that provides one or more wellness indications based on short term heart rate variability (STHRV). STHRV can be conceptualized as heart rate variability that is measured over a short amount of time, as discussed further below. A long term measurement of HRV (e.g., obtained over a measurement time period longer than 24 hours) can fail to provide a high enough HRV sample rate to collect all the data that is possible when determining wellness. An STHRV-based wellness indicator can be provided to a user as a diagnostic, or to another system or process, such as to control delivery of one or more cardiac therapies based on a patient's STHRV.

Among other things, STHRV indicates autonomic balance between the output of the parasympathetic and sympathetic nervous systems, thereby indicating the patient's cardiac condition. Generally, the patient's wellness improves when STHRV increases and worsens when STHRV decreases, unless there is a known reason for STHRV to decrease, such as strenuous activity. An increase in sympathetic tone (which can be associated with strenuous activity) will often result in a decreased STHRV. In some cases, such as if the patient suffers left ventricular dysfunction, the autonomic balance shifts toward the sympathetic nervous system, and the STHRV decreases.

The present STHRV measurements occur over a period of less than 24 hours. Certain examples include STHRV measurements that occur during a period of time compatible with an outpatient procedure, such as a physician office visit that lasts less than one hour. Examples of STHRV measurement time windows can include windows lasting approximately 5 minutes. Certain examples include windows lasting approximately 2 minutes. These windows are not exhaustive or exclusive; other short term time windows are possible. In some examples, the duration of a measurement time window is controlled, such as to allow for window to window comparisons.

Some embodiments measure STHRV over a time window which is related to a therapy. For example, in some embodiments, STHRV measurements take place during administration of a CRT therapy. Some embodiments measure STHRV during a pacing therapy. Other therapies disclosed herein are additionally contemplated.

STHRV measurement duration can also be related to a physical activity. For example, if STHRV is being measured clinically, a care provider can instruct a patient to engage in an activity, and can measure STHRV during the time that activity is performed. In some embodiments, the start and finish of such a measurement window is triggered automatically. In some embodiments, the start and finish are manually controlled. The present subject matter is not limited to clinical embodiments, and can incorporated activity sensors which sense and activity and in turn trigger the measurement of STHRV.

STHRV can be used alone or with other information, such as can be gathered along with STHRV. In various examples, a feedback system adjusts one or more cardiac therapies to increase a patient's STHRV. Various physiological measurements can be acquired in association with one or more corresponding STHRV measurements being acquired. Such physiological measurements can include, but are not limited to, one or more measurements of posture, physical activity, circadian rhythm, a physiological response to one or more therapy alterations, or other measurements. Some examples include a measurement of parameters used in administering one or more therapies.

Knowledge of short term trends can guide short term therapy adjustments. For example, if a patient engages in an activity which is detrimental to wellness, and an STHRV-based wellness indicator in conjunction with a respiration rate monitor, demonstrates that wellness is declining due to the activity, an indicator can be communicated to a patient or caregiver to adjust the activity level. As such, in various examples, multiple STHRV measurements are used in conjunction with one or more physiological measurements taken in association with respective STHRV measurements, such as to initiate or adjust one or more therapies.

STHRV represents the beat-to-beat variance in cardiac cycle length over a “short term” period of time, as discussed above. A “wellness indicator” can be conceptualized as any parameter that uses a measure of the STHRV, including any qualitative expression of the beat-to-beat variance in cardiac cycle length over the short term period of time. In some examples, the wellness indicator represents the time differences between successive cardiac cycle lengths averaged over a specified period of time. In some examples, the cardiac cycle lengths are ventricular cycle lengths, i.e., V-V intervals, or R-R intervals, that are time intervals between successive ventricular depolarizations (R waves). In some examples, the cardiac cycle lengths are atrial cycle lengths, i.e., A-A intervals, or P-P intervals, that are time intervals between successive atrial depolarizations (P waves). In various examples, the wellness indicators include, but are not limited to, the following:

Standard Deviation of Normal-to-Normal intervals (SDNN). SDNN is the standard deviation of the R-R intervals measured over a specified time period during a normal sinus rhythm.

Standard Deviation of Averages of Normal-to-Normal intervals (SDANN). To compute SDANN, R-R intervals during a normal sinus rhythm are measured and averaged over a first time period. The standard deviation of the averaged R-R intervals is computed for a second time period that includes multiple such first time periods. In some examples, measured R-R intervals are averaged over five-minute periods for a time period less than 24 hours. The SDANN is the standard deviation of five-minute mean R-R intervals computed for the time period. In some examples, SDANN includes the standard deviation of average STHRV measured in association with one or more activity levels. Additional examples determine SDANN by monitoring standard deviation of average STHRV measured in association with one or more therapies.

Ratio of LF STHRV to HF STHRV (LF/HF ratio). The LF STHRV generally includes components of the STHRV having frequencies between about 0.04 Hz and 0.15 Hz. The HF STHRV generally includes components of the STHRV having frequencies between about 0.15 Hz and 0.40 Hz. The LF/HF ratio is used to track trends in shifts of autonomic balance. A substantial change in the LF/HF ratio indicates a change in systemic stress that indicates the degree to which the sympathetic nervous system is activated.

STHRV footprint. STHRV footprint can be illustrated as a histogram of the STHRV plotted against heart rate. The time difference between successive R-R intervals are determined for a period of time and plotted versus the heart rate measured over that period of time. In additional embodiments, an STHRV footprint can be determined for the duration of a therapy. Additionally, some embodiments establish an STHRV for the duration of an activity.

Root-Mean-Square of Successive Differences (RMSSD). Root-mean-square values are computed, each for time differences between successive R-R intervals determined for a period of time.

Normal-to-Normal 50 ms (NN50). The number of interval differences of successive normal-to-normal intervals greater than 50 ms (or other specified value).

Proportion Normal-to-Normal 50 ms (NN50). The proportion derived by dividing NN50 by the total number of NN intervals.

Root-Mean-Square of Successive Differences (RMSSD). Root-mean-square values are computed, each for time differences between successive R-R intervals determined for a period of time.

The above examples of wellness indicators can be measured for providing a diagnostics. In some examples, such wellness indicators are part of a feedback system that adjusts one or more cardiac therapies. Some embodiments use wellness indicators to alter electrical pulse therapy. Some embodiments use the wellness indicators to provide an activity adjustment indication. Such an indication can provide information to a patient, and in some cases to a care provider, that the patient's physical activity should be altered. These examples do not provide an exhaustive or exclusive list of wellness indicators that may be used, and other parameters capable of representing or indicating the STHRV are possible.

In various examples, one or more therapies are delivered to a patient with different specified values of one or more therapy parameters (such as a first cardiac resynchronization therapy (CRT) pacing parameter and a second CRT pacing parameter) to evaluate the effect of the different values of the one or more therapy parameters on STHRV. One or more wellness indicators are measured during the evaluation. The value of one or more therapy parameters that yield the most desirable STHRV can be then specified as values for later use with the patient. This is one example of how STHRV and wellness indicators can be used to monitor or improve wellness.

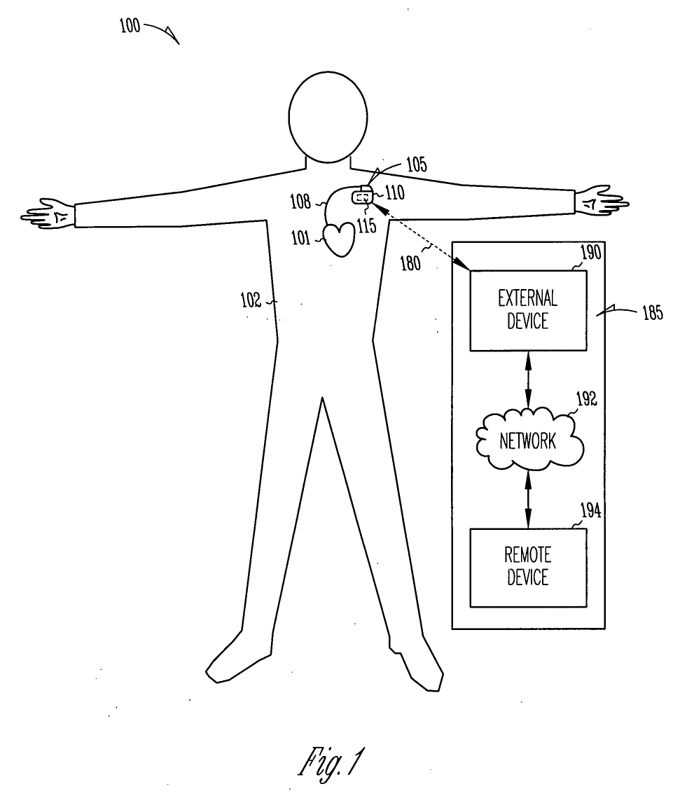

FIG. 1 is an illustration of an example of a CRM system 100 and portions of an environment in which CRM system 100 is used. In this example, the CRM system 100 includes an implantable system 105, an external system 185, and a telemetry link 180 providing for communication between implantable system 105 and external system 185.

Implantable system 105 includes, among other things, implantable medical device 110 and lead system 108. In various examples, implantable medical device 110 is an implantable CRM device including one or more of a pacer, a cardioverter/defibrillator, a CRT device, a cardiac remodeling control therapy (RCT) device, a neurostimulator, a drug delivery device or a drug delivery controller, and a biological therapy device.

As illustrated in FIG. 1, implantable medical device 110 is implanted in a body 102. In various examples, lead system 108 includes implantable electrodes for sensing one or more physiological signals. Lead system 108 additionally includes one or more implantable electrodes, such as for delivering one or more pacing pulses, one or more cardioversion/defibrillation shocks, one or more neurostimulation pulses, one or more or pharmaceutical or other substances in certain examples. In some examples, lead system 108 includes one or more pacing-sensing leads each including at least one electrode placed in or on a heart 101 for sensing electrogram or delivering pacing pulses. In other examples, lead system 108 includes one or more neurostimulation-sensing leads each including at least one electrode placed on a nerve of the autonomic nervous system for sensing neural signals and delivering neurostimulation pulses. In other examples, lead system 108 includes one or more pacing-sensing leads and one or more neurostimulation-sensing leads to synchronize neurostimulation with intrinsic activities of heart 101 or pacing.

In some examples, external system 185 is a patient management system including a local external device 190, a network 192, and a remote device 194. Local external device 190 is within the vicinity of implantable medical device 110 and communicates with implantable medical device 110 bi-directionally via telemetry link 180. Remote device 194 is in a remote location and communicates with external device 190 bi-directionally via network 192, thus allowing a user to monitor and treat a patient from a distant location. In other examples, external system 185 includes a programmer communicating with implantable medical device 110 bi-directionally via telemetry link 180.

The distribution of monitoring system 115 in system 100 can vary. In some examples, as illustrated in FIG. 1, implantable medical device 110 includes the entire system 115. This allows implantable system 105 to monitor for heart failure without communicating to external system 185. In other examples, implantable medical device 110 and external system 185 each include portions of system 115. Heart failure information is collected when implantable medical device 110 and external system 185 are communicatively coupled via telemetry link 180.

In some examples, sensing and stimulation occur outside the body. The present technology is not limited to implanted bodies, and can be extended to examples in which sensors and stimulators are applied outside a body. In some of these examples, sensors and stimulators are controlled by electronics that are not implanted.

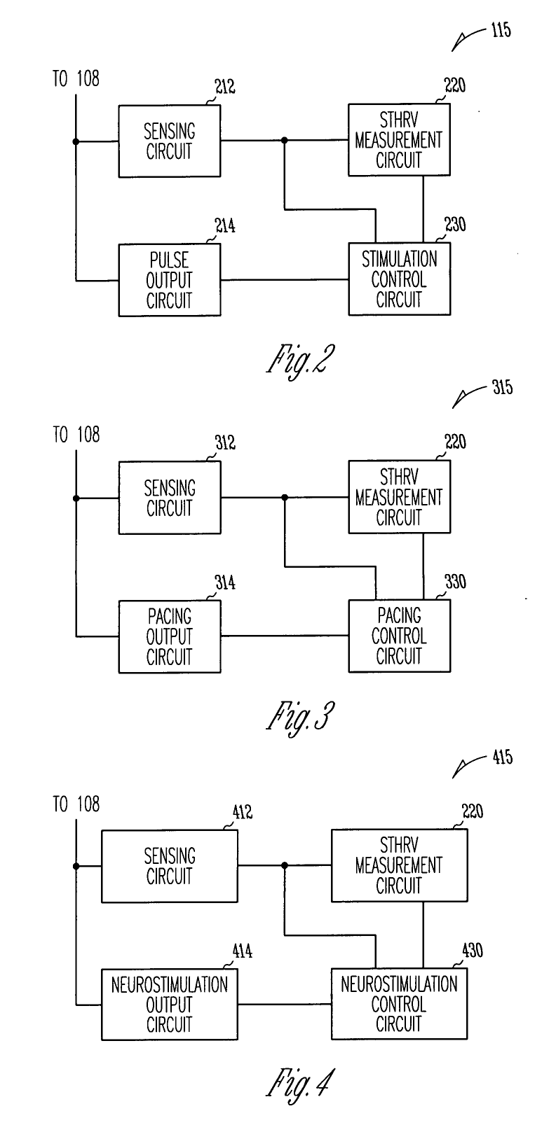

FIG. 2 is a block diagram illustrating some examples of a sensing system including an optional stimulation system 115. The system 115 includes a sensing circuit 212, an optional pulse output circuit 214, an STHRV measurement circuit 220, and an optional stimulation control circuit 230.

Circuits of the present examples may be implemented using any combination of hardware and software. For example, one or more elements may be implemented using an application-specific circuit constructed to perform one or more particular functions. Elements are implemented using firmware in various examples. Some examples use a general-purpose circuit programmed to perform such function(s). Such a general-purpose circuit includes, but is not limited to, a microcontroller or portions thereof, and a programmable logic circuit or a portion thereof. The controller can include a digital signal controller, or other processing components, and may be integrated into a single component or partitioned into more than one component.

The sensing circuit 212 senses at least a cardiac signal that allows for a measurement of the STHRV. In some examples, the sensing circuit 212 senses one or more additional signals each indicative of one or more cardiac functions from the heart and the autonomic nervous system through one or more electrodes of lead system 108.

The optional pulse output circuit 214 delivers electrical stimulation pulses to the heart and the autonomic nervous system through one or more electrodes of lead system 108. The STHRV measurement circuit 220 measures the STHRV and produces at least one wellness indicator based on a signal sensed by sensing circuit 212. In various examples, STHRV measurement circuit 220 includes, but is not limited to, one or more of an SDNN circuit to provide an SDNN, an SDANN circuit to provide an SDANN, an LF/HF ratio circuit to provide an LF/HF ratio, an STHRV footprint circuit to provide an STHRV footprint, and an RMSSD circuit to provide an RMSSD.

In some examples, the STHRV measurement circuit 220 includes an autonomic balance monitor to monitor a wellness indicator indicative of a balance between sympathetic and parasympathetic activities. In some examples, the autonomic balance monitor includes the LF/HF ratio circuit to provide the LF/HF ratio as the wellness indicator indicative of the balance between sympathetic and parasympathetic activities.

In some examples, the STHRV measurement circuit 220 is configured to store one or more STHRV measurements. Some examples include a comparator circuit which compares a first measured STHRV to a threshold STHRV to determine a first wellness indicator. A threshold STHRV can be a STHRV that has been determined through study to be indicative of a desired level of wellness. Additional examples compare a first measured STHRV to a second STHRV to provide an STHRV trend. Some examples include trending the first wellness indicator and the second wellness indicator to provide an STHRV wellness trend.

The optional stimulation control circuit 230 controls the delivery of the electrical stimulation pulses from optional pulse output circuit 214 using one or more stimulation parameters that are adjusted or improved based at least in part on the wellness indicator. In some examples, optional stimulation control circuit 230 determines a suitable value for each adjustable parameter that affects the STHRV.

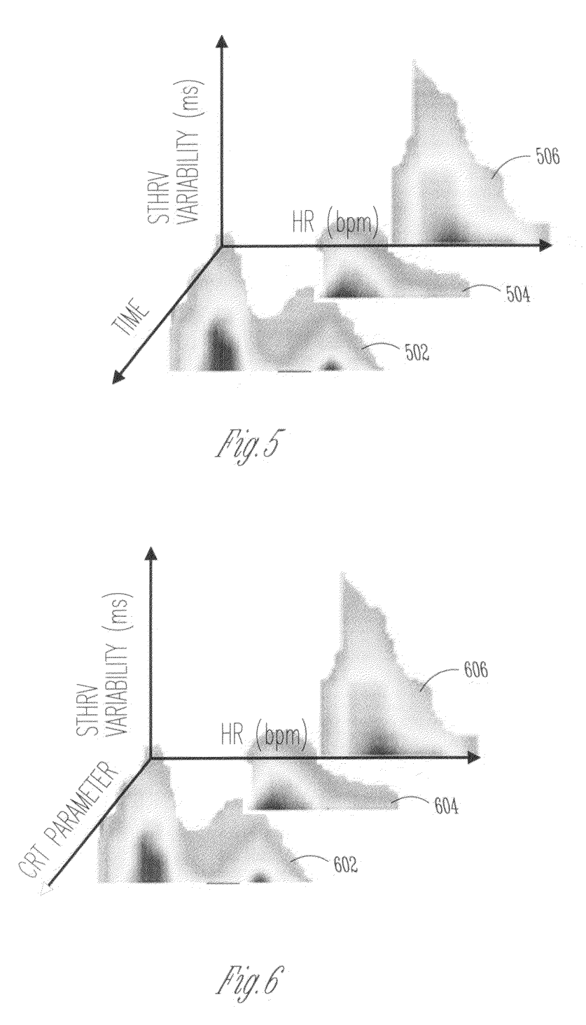

FIG. 3 is a block diagram illustrating some examples of a pacing system 315. Pacing system 315 includes a sensing circuit 312, a pacing output circuit 314, STHRV measurement circuit 220, and a pacing control circuit 330.

Sensing circuit 312 generally includes an electrogram sensing circuit. The electrogram sensing circuit senses one or more atrial and ventricular electrograms. Pacing output circuit 314 delivers pacing pulses to one or more atrial and ventricular sites. Pacing control circuit 330 controls the delivery of the pacing pulses from pacing output circuit 314 using one or more pacing parameters that are adjusted or improved based on the wellness indicator. In some examples, pacing control circuit 330 determines an improved value for each adjustable pacing parameter that affects the STHRV. Examples of such adjustable pacing parameters include, but are not limited to, atrioventricular delays (“AVDs”) and interventricular delays (“VVDs”), and pacing sites (sites to which the pacing pulses are delivered, e.g., electronic repositioning).

FIG. 4 is a block diagram illustrating some examples of a neurostimulation circuit 415. Neurostimulation system 415 includes a sensing circuit 412, a neurostimulation output circuit 414, STHRV measurement circuit 220, and a neurostimulation control circuit 430.

Sensing circuit 412 includes a neural signal sensing circuit in addition to an electrogram sensing circuit. The neural sensing circuit senses one or more neural signals from the autonomic nervous system including sympathetic and parasympathetic nerves. The electrogram sensing circuit senses one or more atrial and ventricular electrograms to allow for measurement of the STHRV. In some examples, the one or more atrial and ventricular electrograms allow for a delivery of neurostimulation that is synchronized to one or more cardiac activities detectable from the one or more electrograms.

Neurostimulation output circuit 414 delivers neurostimulation pulses to one or more nerves of the autonomic nervous system. Neurostimulation control circuit 430 controls the delivery of the neurostimulation pulses from neurostimulation output circuit 414 using one or more neurostimulation parameters that are adjusted or improved based at least in part on the wellness indicator. In some examples, neurostimulation control circuit 430 determines an improved value for each adjustable pacing parameter that affects the STHRV. Examples of such adjustable neurostimulation parameters include, but are not limited to, one or more of stimulation frequencies, stimulation amplitudes, and stimulation sites (sites to which the neurostimulation pulses are delivered).

In some examples, stimulation system 115 includes a combination of pacing system 315 and neurostimulation system 415, and implantable medical device 110 includes an implantable pacemaker-neurostimulator. In this example, sensing circuit 212 combination of sensing circuits 312 and 412, pulse output circuit 214 includes a combination of pacing output circuit 314 and neurostimulation circuit 414, and stimulation control circuit 230 includes a combination of pacing control circuit 330 and neurostimulation circuit 430. In some examples, stimulation system 115 delivers pacing and neurostimulation pulses in a temporally coordinated manner, such as to adjust or improve the wellness indicator.

In the following examples, examples of stimulation system 115, including pacing system 315, neurostimulation system 415, and the combination thereof, are discussed to illustrate, but not to restrict, the use of a wellness indicator for stimulation control according to the present subject matter.

Because STHRV is indicative of a patient's cardiac condition, an STHRV-based wellness indicator is capable of indicating one or more effects of an electrical stimulation therapy including pacing therapy, autonomic neurostimulation therapy, and a combination of the pacing and autonomic neurostimulation therapies. To increase the benefit of the therapy, one or more therapy parameters are initiated or adjusted for an improved STHRV, such as can be achieved by delivering electrical stimulation pulse to the patient's heart and autonomic nervous system.

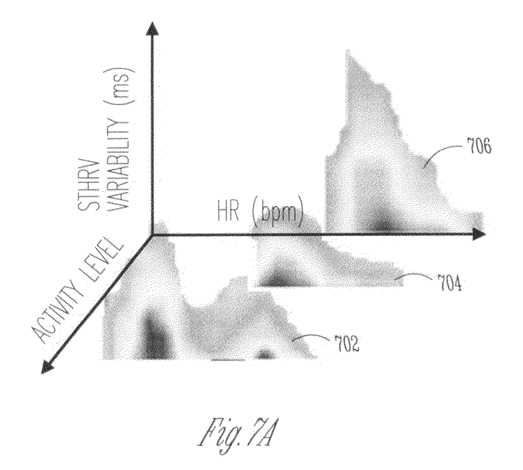

FIG. 5 illustrates multiple STHRV measurements associated with time. The illustration shows a first STHRV footprint 502, a second STHRV footprint 504, and a third STHRV footprint 506. The first STHRV footprint correlates to a first time of day. The second STHRV footprint correlates to a second time of day. The third STHRV footprint correlates to a third time of day. In various examples, the first, second, and third times of day occur during the same 24 hour period. In various examples, the three STHRV footprints can be compared to one another and cross-referenced with the recorded times of day to demonstrate useful information. While three STHRV footprints are illustrated, the present application is not so limited, and one or more STHRV footprints are possible.

For example, some patients suffer from a disease where their sympathetic tone is not sufficiently diminished during sleep. A long term HRV system would not detect that a patient suffers from such a disease, as it incorporates data that is spread over a 24 hour period. At least two STHRV footprints can be compared to one another. In some examples, a first STHRV footprint that is developed during a first portion of the day when a patient is awake and at rest is compared to second STHRV footprint that is developed during a second portion of the day when the patient is sleeping. If the size of the second STHRV footprint is not appropriately reduced when compared to the first STHRV footprint, an indication can be provided that the patient is potentially or actually suffering from a disease in which sympathetic tone is not diminished during sleep.

In some examples, an implantable device detects sleep apnea by measuring one or more respiration parameters and associating the one or more respiration parameters with at least a first wellness indicator and a second wellness indicator. In some of these examples, circadian rhythm is monitored in addition to STHRV and respiration.

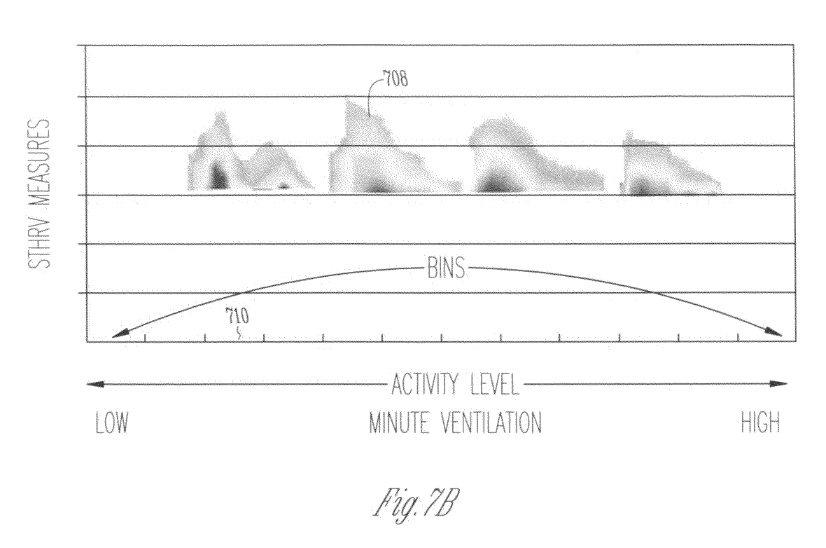

FIG. 6 illustrates multiple STHRV measurements associated with multiple CRT parameters. The illustration shows a first STHRV footprint 602, a second STHRV footprint 604, and a third STHRV footprint 606. The first STHRV footprint correlates to a first CRT parameter. The second STHRV footprint correlates to a second CRT parameter. The third STHRV footprint correlates to a third CRT parameter. In various examples, therapies based on the first, second, and third CRT parameters are administered during the same 24 hour period. In additional examples, the time period is compatible with an outpatient procedure. In various examples, the three STHRV footprints can be compared to one another and cross-referenced with the recorded therapy parameters to demonstrate useful information. While three STHRV footprints are illustrated, the present application is not so limited, and one or more STHRV footprints are possible.

For example, a stimulation control circuit can compare the first STHRV footprint 602, the second STHRV footprint 604, and the third STHRV footprint 606 and determine which CRT parameter presents an improved STHRV footprint size. A stimulation control circuit can then alter therapy according to that determination and administer therapy using the improved CRT parameter. If a first CRT parameter correlates with one or more of a plurality of other parameters, such as time of day, activity, or another parameter, to demonstrate an improved STHRV during that correlation, a stimulation control circuit can administer a first CRT pacing therapy when those parameters are present, and can administer another CRT pacing therapy when other parameters are recognized.

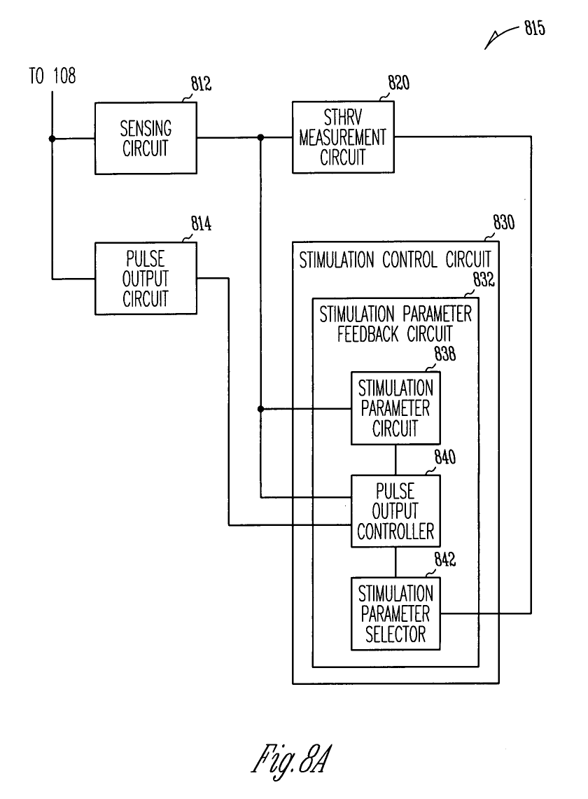

FIG. 7A illustrates multiple STHRV measurements associated with multiple activity levels. The illustration shows a first STHRV footprint 702, a second STHRV footprint 704, and a third STHRV footprint 706. The first STHRV footprint correlates to a first activity level. The second STHRV footprint correlates to a second activity level. The third STHRV footprint correlates to a third activity level. In various examples, the first, second and third activity levels occur during the same 24 hour period. In various examples, the time period is compatible with an outpatient procedure. In various examples, the three STHRV footprints can be compared to one another and cross-referenced with the recorded activity levels to demonstrate useful information. While three STHRV footprints are illustrated, the present application is not so limited, and one or more STHRV footprints are possible.

For example, a stimulation control circuit can compare the first STHRV footprint 702, the second STHRV footprint 704, and the third STHRV footprint 706 and evaluate which activity levels result in decreased wellness. In some examples, if a stimulation control circuit determines that a particular activity results in decreased wellness, it can produce an activity adjustment indicator to alert a patient or a caregiver that the activity that is causing decreased wellness should be avoided. In some examples, the activity adjustment indicator is an audible signal produced by an implantable device that a patient perceives. In additional examples, the activity adjustment indicator is an activity adjustment indicator that is readable by a caregiver using a programmer to communicate with an implantable device or with an external measurement system. Other configurations are possible.

FIG. 7B illustrates multiple STHRV measurements associated with respiration and multiple activity levels. In various examples, respiration is measured by a minute ventilation or other respiration sensor. Minute ventilation or another indication of respiration is then associated with an STHRV measurement. A wellness indicator, such as the illustrated footprint 708, is then associated with the respiration indication. In various examples, the respiration indication, and the associated wellness indicator, are disposed in one of a plurality of activity level groups or bins, such as bin 710. If the wellness indicator, the respiration indicator, or another measurement indicates decreased wellness, an activity adjustment indicator may be administered. In some examples, an activity adjustment indicator is displayed on a programmer or another device that a caregiver or patient may perceive. An activity adjustment indicators include, but are not limited to, indicators transmitted to a programmer, indicators audible or otherwise perceivable by a patient having an implantable device, and other indicators. Examples in which a therapy parameter is altered as disclosed herein are additionally possible.

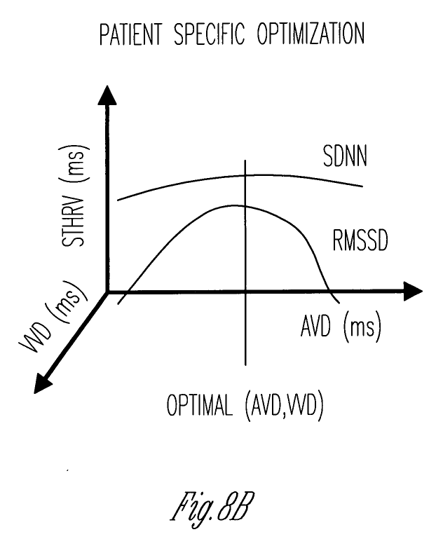

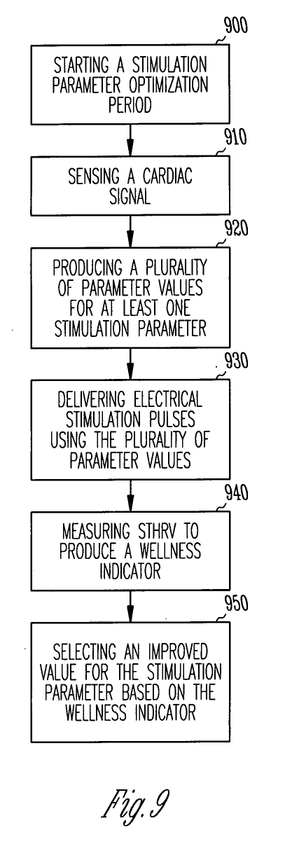

FIG. 8 is a block diagram illustrating some examples of a stimulation system 815 that includes an STHRV-based stimulation parameter feedback circuit. Stimulation system 815 includes sensing circuit 812, pulse output circuit 814, STHRV measurement circuit 820, and a stimulation control circuit 830. Stimulation control circuit 830 includes a stimulation parameter feedback circuit 832 that adjusts at least one stimulation parameter to an improved value based on at least one wellness indicator produced by STHRV measurement circuit 820.

In some examples, stimulation control circuit 830 includes a pacing control circuit that includes a pacing parameter feedback circuit to adjust at least one pacing parameter to an improved value based on the wellness indicator. The pacing parameter feedback circuit includes, but is not limited to, one or more of an AVD feedback circuit to improve an AVD, a VVD feedback circuit to improve a VVD, and a pacing site feedback circuit to improve a selection of one or more sites to which the cardiac pacing pulses are delivered. In general, the pacing parameter feedback circuit allows feedback of a pacing parameter whose value affects the STHRV by adjusting that parameter for an improved STHRV indicated by the wellness indicator. Other control circuits that work with the present system include, but are not limited to, a neurostimulation control circuit, a CRT pacing control circuit, an RCT pacing control circuit, and other therapy circuits.

In some examples, STHRV measurement circuit 820 recurrently or continuously updates the wellness indicator to reflect changes in the patient's cardiac condition, and stimulation parameter feedback circuit 832 adjusts the stimulation parameter to the improved value based on the wellness indicator on an ongoing basis. In other examples, stimulation parameter feedback circuit 832 determines the improved value for the stimulation parameter based on the wellness indicator during a stimulation parameter feedback period. This period can be started according to a specified schedule, such as on a programmed periodic basis, or is started in response to a command, such as a command entered by a caregiver. Stimulation parameter feedback circuit 832 includes a stimulation parameter circuit 838, a pulse output controller 840, and a stimulation parameter selector 842.

In some examples, stimulation parameter circuit 838 includes a physiological parameter measurement circuit that measures at least one physiological parameter related to a patient's cardiac condition. Examples of the physiologic parameter include the heart rate and a time interval between two detectable cardiac electrical and mechanical events. In various examples stimulation parameter circuit 838 produces the plurality of values for the stimulation parameter based on the measured physiological parameter.

Stimulation parameter circuit 838 produces a plurality of parameter values for the stimulation parameter that is to be improved. Pulse output controller 840 controls the delivery of electrical stimulation pulses using the plurality of parameter values during the stimulation parameter feedback period. Stimulation parameter selector 842 selects an improved value for the stimulation parameter from the plurality of parameter values. The improved value is the value corresponding to an improved value of the wellness indicator obtained with pacing using the plurality of parameter values.

When two or more stimulation parameters are to be improved, stimulation parameter circuit 838 produces a plurality of values for each stimulation parameter. Pulse output controller 840 controls the delivery of a plurality of series of stimulation pulses. Each series of stimulation pulses is delivered using a combination of values produced for all the stimulation parameters to be improved. Stimulation parameter selector 842 selects an improved combination of values for all the stimulation parameters to be improved. The improved combination of values is the combination of values corresponding to a improved value of the wellness indicator produced for the stimulation parameter feedback period.

FIG. 8B is a three dimensional graph illustrating improved CRT pacing based on STHRV measurements. The graph shows an SDNN curve and an RMSSD curve, each of that are wellness indicators. An optimal pacing mode based in light of these wellness indicators is illustrated. The pacing mode was discovered by adjusting AVD and VVD. The graph in FIG. 8B is one example of how at least a first and second therapy are provided, and a trend is determined based on the effects of the first and second therapy. In various examples, such trends are used to determine a therapy which offers improved wellness. That therapy is then selected for patient treatment.

For example, associating a first electrical stimulation therapy with the first wellness indicator is performed in some embodiments. The first electrical stimulation therapy is based on one or more stimulation parameters. The electrical stimulation therapy can be any of those disclosed herein, including, but not limited to, CRT therapy and neurostimulation therapy. The example includes measuring a second STHRV to provide a second wellness indicator. Associating a second electrical stimulation therapy with the second wellness indicator is included in the examples. The example also includes trending the first wellness indicator and the second wellness indicator to provide an STHRV wellness trend. Determining an improved electrical stimulation based on the STHRV wellness trend is included in the example. The example also includes providing the improved electrical stimulation therapy.

The present disclosure is not limited to trending the efficacy of a first therapy versus a second therapy. Trends are also used, in some examples, to compare the wellness realized during a first activity versus a second activity. Trends are used, in some examples, to study changes to STHRV over the course of time, and to diagnose a disorder based on the STHRV trend. Some examples compare the STHRV trend to a specified trend. Some examples preprogram a device with such a specified trend, and automatically provide therapy based on the comparison of the measured STHRV trend to the specified trend.

FIG. 9 is a flow chart illustrating some examples of a method for stimulation parameter feedback using a wellness indicator. In some examples, the method is performed by stimulation system 815.

A stimulation parameter feedback period is started at 900. This starts the process of optimizing at least one stimulation parameter as illustrated in FIG. 9. The stimulation parameter feedback period lasts until the process is completed. The stimulation parameter includes, but not limited to, an AVD, a VVD, pacing sites, neurostimulation pulse frequency, and neurostimulation sites.

A signal indicative of a cardiac function is sensed at 910. The signal includes one or more of an atrial electrogram, a ventricular electrogram, a neural signal indicative of sympathetic neural activities, and a signal indicative of parasympathetic neural activities. At least one cardiac signal allowing for measurement of a wellness indicator is sensed.

A plurality of parameter values for the stimulation parameter is produced at 920. In some examples, a physiologic parameter related to a patient's cardiac condition is measured at the beginning of the stimulation parameter feedback period. The plurality of parameter values are calculated based on the physiological parameter. In other examples, a physiologic parameter related to a patient's cardiac condition is monitored throughout the stimulation parameter feedback period. The value for the stimulation parameter is dynamically calculated as a function of the physiological parameter, which changes dynamically during the stimulation parameter feedback period.

Electrical stimulation pulses are delivered using the plurality of parameter values at 930. In some examples, pacing pulses are delivered. In other examples, neurostimulation pulses are delivered. In other examples, pacing and neurostimulation pulses are delivered in a temporally coordinated manner. The electrical stimulation pulses are delivered by engaging a stimulation circuit during the stimulation parameter feedback period. The stimulating circuit uses the stimulation parameter that is to be improved. Examples of the stimulation circuit include, but are not limited to, a bradycardia pacing circuit, a CRT pacing circuit, an RCT pacing circuit, an autonomic neurostimulation circuit, and a combined pacing-neurostimulation circuit.

The STHRV is measured based on the sensed cardiac signal, and at least one wellness indicator is produced based on the STHRV measurement, at 940. In some examples, the sensed cardiac signal is an atrial electrogram, and the wellness indicator is produced based on atrial intervals measured from the atrial electrogram. In other examples, the sensed cardiac signal is a ventricular electrogram, and the wellness indicator is produced based on ventricular intervals measured from the ventricular electrogram.

An improved parameter value for the stimulation parameter is selected from the plurality of parameter values produced during the stimulation parameter feedback period at 950. The improved parameter value corresponding to a improved value of the wellness indicator produced for the stimulation parameter feedback period.

It is to be understood that the above detailed description is intended to be illustrative, and not restrictive. Other embodiments, including any possible permutation of the system components discussed in this document, will be apparent to those of skill in the art upon reading and understanding the above description. The scope of the invention should, therefore, be determined with reference to the appended claims, along with the full scope of equivalents to which such claims are entitled.

What is claimed is:

1. A cardiac rhythm management system, comprising:
a sensing circuit configured to sense a cardiac signal;
a short term heart rate variability (STHRV) measurement circuit, coupled to the sensing circuit, configured to characterize the cardiac signal to measure at least a first STHRV measurement during a period of time compatible with an outpatient procedure and to provide at least a first wellness indicator based on the first STHRV measurement; and
a wellness circuit configured to compute a first wellness indicator using the first STHRV measurement.

2. The system of claim 1, comprising:
a communications circuit coupled to the wellness circuit; and
a programmer configured to communicate with the communications circuit and to display the wellness indicator.

3. The system of claim 1, wherein the sensing circuit comprises a circadian rhythm measurement circuit to associate the first wellness indicator with a circadian rhythm parameter.

4. The system of claim 1, wherein the STHRV measurement circuit comprises one or more of a Standard Deviation of Normal-to-Normal intervals (SDNN) circuit to provide an SDNN characterizing the STHRV measurement, a Standard Deviation of Averages of Normal-to-Normal intervals (SDANN) circuit to provide an SDANN characterizing the STHRV measurement, a Ratio of Low Frequency STHRV to High Frequency STHRV ratio (LF/HF ratio) circuit to provide an LF/HF ratio characterizing the STHRV measurement, an STHRV footprint circuit to provide an STHRV footprint characterizing the STHRV measurement, and a Root-Mean-Square of Successive Differences (RMSSD) circuit to provide an RMSSD characterizing the STHRV measurement.

5. The system of claim 1, comprising:
a pulse output circuit configured to deliver electrical stimulation pulses; and
a stimulation control circuit coupled to the sensing circuit, the STHRV measurement circuit, and the pulse output circuit, the stimulation control circuit being configured to control delivery of one or more electrical stimulation pulses based on at least the first wellness indicator.

6. The system of claim 5, wherein the stimulation control circuit is configured to control delivery of cardiac resynchronization therapy (CRT) pacing.

7. The system of claim 5, wherein the stimulation control circuit includes a feedback circuit configured to adjust delivery of the one or more electrical stimulation pulses based on the first wellness indicator and at least a second wellness indicator.

8. The system of claim 1, wherein the period of time is approximately 5 minutes.

9. The system of claim 8, wherein the period of time is approximately 2 minutes.

10. A method, comprising:
sensing a cardiac signal; and
measuring STHRV to provide at least a first wellness indicator based on a first STHRV measured during a time period compatible with an outpatient procedure.

11. The method of claim 10, comprising:
storing the first STHRV; and
comparing the first STHRV to a threshold STHRV to determine the first wellness indicator.

12. The method of claim 10, wherein measuring STHRV to provide the wellness indicator comprises measuring STHRV to provide an autonomic balance parameter indicative of a balance between sympathetic and parasympathetic activities.

13. The method of claim 10, comprising detecting sleep apnea by measuring a first respiratory parameter and a second respiratory parameter and associating the first respiratory parameter with the first wellness indicator and a the second respiratory parameter with a second wellness indicator.

14. The method of claim 10, comprising measuring a first posture and a second posture, and associating the first postures with the first wellness indicator, and the second posture with a second wellness indicator.

15. The method of claim 10, comprising measuring a second STHRV to provide a second wellness indicator and trending the first wellness indicator and the second wellness indicator to provide an STHRV wellness trend.

16. The method of claim 15, comprising measuring the first STHRV and the second STHRV under substantially similar conditions; and indicating a potential heart failure condition when the STHRV wellness trend demonstrates a decrease in STHRV over time.

17. The method of claim 16, comprising measuring a third STHRV to provide a third wellness indicator and providing an indication of a potential myocardial infarction when the first and third wellness indicators are substantially similar, while the second wellness indicator, that is measured between the first and third wellness indicators, demonstrates a wellness indicator that is less than the first and third wellness indicators.

18. The method of claim 10, comprising:
detecting a first activity level and associating the first activity level with the first wellness indicator;
detecting a second activity level and associating the second activity level with a second wellness indicator; and
detecting a decrease in wellness when the second wellness indicator is less than the first wellness indicator.

19. The method of claim 18, comprising providing an activity adjustment indicator when a decrease in wellness is detected.

20. The method of claim 10, comprising providing electrical stimulation pulses based at least on the first wellness indicator.

21. The method of claim 20, comprising:
associating a first electrical stimulation therapy with the first wellness indicator;
measuring a second STHRV to provide a second wellness indicator;
associating a second electrical stimulation therapy with the second wellness indicator;
trending the first wellness indicator and the second wellness indicator to provide an STHRV wellness trend;
determining an improved electrical stimulation based on the STHRV wellness trend; and
providing the improved electrical stimulation therapy.

22. The method of claim 20, comprising:
sensing a neural signal;
associating the first wellness indicator with the neural signal; and
delivering neurostimulation therapy based on the association of the first wellness indicator with the neural signal.

23. A system, comprising:
an implantable case including at least a first lead, the first lead having at least a first electrode;
means for sensing a cardiac signal, the means for sensing a cardiac signal being disposed in the implantable case and being in electrical connection with at least the first electrode;
means for measuring STHRV to provide at least one wellness indicator based on a cardiac signal measured during an outpatient procedure; and
means for indicating wellness based on associating at least the first wellness indicator with at least a first wellness state.

24. The system of claim 23, wherein the means for sensing the cardiac signal include a sensing circuit configured to sense a cardiac signal.

25. The system of claim 24, wherein the means for measuring STHRV to provide at least one wellness indicator based on the cardiac signal during an outpatient procedure include a short term heart rate variability (STHRV) measurement circuit, coupled to the sensing circuit, and configured to characterize the cardiac signal to measure at least a first STHRV measurement during a period of time compatible with an outpatient clinical visit and to provide at least a first wellness indicator based on the first STHRV measurement.
